(19) United States
(12) Patent Application Publication
(10) Pub. No.: US 2023/0058316 A1
(43) Pub. Date: Feb. 23, 2023

(54) PEGYLATED IGE-DEPENDENT HISTAMINE-RELEASING FACTOR (HRF)-BINDING PEPTIDE AND USE THEREOF

(71) Applicant: EWHA UNIVERSITY - INDUSTRY COLLABORATION FOUNDATION, Seoul (KR)

(72) Inventors: Kyunglim LEE, Seoul (KR); Hyeran SEO, Seoul (KR); Haejun PYUN, Seoul (KR)

(73) Assignee: EWHA UNIVERSITY - INDUSTRY COLLABORATION FOUNDATION, Seoul (KR)

(21) Appl. No.: 17/793,369

(22) PCT Filed: Feb. 1, 2021
(86) PCT No.: PCT/KR2021/001282

§ 371 Date: Jul. 15, 2022

(30) Foreign Application Priority Data
Feb. 7, 2020 (KR) 10-2020-0014941

(51) Int. Cl. A61K 47/60

(52) U.S. Cl. CPC A61K 47/60

(57) ABSTRACT

The present invention relates to a modified HRF binding peptide. The modified HRF-binding peptide according to the present invention can exert drug efficacy with high stability in vivo, can be effectively used as a drug because the number of administrations can be reduced due to its longer half-life, and can prevent or treat allergies, malaria, autoimmune diseases, acute or chronic inflammatory diseases, hypertension, and cancer in humans as well as animals by effectively inhibiting histamine secretion in cells.

### BACKGROUND OF THE INVENTION

### 1. Field of the Invention

The present invention relates to PEGylated IgE-dependent histamine releasing factor (HRF) binding peptides and uses thereof.

### 2. Description of the Related Art

Inflammatory disease means to a pathological condition caused by chronic or acute inflammation and can be caused by abnormalities in the immune system. Inflammation is the expression of a normal and protective in vivo defense mechanism that appears locally against tissue damage caused by physical trauma, harmful chemicals, microbial infection, or irritating substances in metabolites in vivo. Such inflammation is triggered by various chemical mediators produced from damaged tissues and migrating cells, and these chemical mediators are known to vary depending on the type of inflammatory process. In a normal case, the living body neutralizes or removes the onset factor through inflammatory response and regenerates damaged tissue to restore normal structure and function, but if not, inflammation may progress to a disease state such as chronic inflammation. In addition, when inflammation is inappropriately triggered by innocuous substances such as pollen or by autoimmune reactions such as asthma, rheumatoid arthritis, and psoriasis, the protective reaction itself rather damages the tissue, so a preventive or therapeutic agent for inflammatory diseases is required. Inflammatory reactions can be observed in almost all clinical diseases, and among these inflammatory diseases, there are bacterial diseases that can be causally treated by administration of antibiotics. However, in most cases, the onset is due to tissue damage caused by an autoimmune reaction, so it is known as an incurable disease without a specific treatment.

An example of an inflammatory disease is an allergic disease. Allergic diseases include atopic dermatitis, asthma, allergic bronchiectasis, hay fever, conjunctivitis, rhinitis, dermatitis, eczema, urticaria, hives, allergy caused by food, atmosphere, drug, pollen, mold, dust and animal, serum sickness, and anaphylactic shock. These diseases may appear alone or multiple diseases may appear at the same time, and in general, immune hypersensitivity inflammatory diseases including atopic dermatitis are representative examples. Until now, the exact cause of the onset of immune hypersensitivity inflammatory diseases, including atopic dermatitis, is not known, but it is generally assumed that genetic and immunological factors are involved, and that other environmental and psychological factors act as exacerbation factors.

Currently, the most widely used treatment for immune hypersensitivity inflammatory disease including atopic dermatitis is dexamethasone, which is known as a steroid. However, this is only effective for short-term treatment, and when long-term treatment for more than 1 year, stability and efficacy are not established, and there are reports of side effects such as skin thinning, skin atrophy, scarring, and skin discoloration (Gil-Ran Shin, 2009, Daejeon University Thesis, 13).

Malaria is a very serious and complicated disease that threatens human health in the 21^{st} century. Worldwide, approximately 3 million people become infected with malaria, and 15 million die each year. Malaria is an infectious disease caused by four types of malarial parasites (Plasmodium), and among them, Plasmodium falciparum is the most serious. Malaria is emerging as a serious problem in developing countries, especially in Africa. When infected with a malaria strain, red blood cells become malformed, and when they accumulate on the walls of blood vessels, they block blood flow, leading to complications in the brain, kidney, liver, etc. Accordingly, insecticides and antimalarial agents for controlling mosquitoes, which are sources of infection, are being developed. And, a paper has been reported that some of the antimalarial agents are effective by binding to TCTP, that is, HRF. For example, it has been reported that artemisinin is effective by binding to HRF. However, the occurrence of malaria tends to increase due to the increase in resistance of mosquitoes to existing insecticides and the emergence of strains resistant to antimalarial agents. Moreover, as the possibility of malaria occurring outside of malaria outbreak regions due to global warming and the like increases, the development of new mosquito insecticides and antimalarial agents is urgently required.

It is known that allergy is caused by the genetically hypersensitive IgE production to allergens, or the disruption of the balance between IL-4 (Interleukin-4), which increases the secretion of IgE, and y-interferon, which decreases the secretion of IgE. Once exposed to an allergen, an immediate reaction occurs, various cells involved in inflammation are gathered. A few hours later, a late-phase reaction (hereinafter referred to as “LPR”) occurs by histamine and various cytokines secreted from basophils, eosinophils and lymphocytes. In LPR, histamine is secreted from basophils, and at this time, there is no allergen that initially initiated the reaction, so what triggers histamine release from basophils was of interest. In addition, only about half of allergic patients progress to LPR, and the causative agent was of great interest. It has been known that cytokines such as MCP-3, MCP-1 or RANTES secrete histamine. However, in IgE-dependent LPR, it was found that only a substance called IgE-dependent histamine-releasing factor (hereinafter referred to as “HRF”) can release histamine from basophils (MacDonald et al., 1995).

HRF is a protein consisting of 172 amino acids present in all cytoplasm, known as translationally controlled tumor protein (TCTP) (Bohm et al., 1989). Among the amino acids, 45 amino acids at the C-terminus form a basic domain, and this region exhibits about 46% homology with MAP-1B, a microtubule-associated protein. Therefore, this region is presumed to be a microtubule binding protein. The present inventors confirmed that HRF can cross the cell membrane despite it is a hydrophilic protein, and first identified the fact that HRF present in cells binds to CD3 (cytoplasmic domain 3), a large cytoplasmic loop in (Na,K)ATPase alpha subunit, through Korean Patent 10-0457350. The present inventors also demonstrated that hypertension was induced in transgenic mice overexpressing HRF intracellularly. On the other hand, although HRF is a protein present in the cytoplasm, McDonald et al. (1995) found HRF outside the cell. It is also known that HRF stimulates IgE-sensitized basophils to liberate histamine while present outside the cell.

However, although HRF is already well known as the most important mediator of late-stage allergic disease, the development of a distinct therapeutic agent has not been progressed because the exact mechanism of action is not disclosed. That is, in the case of HRF in the form of a recombinant monomer, it is not clearly known which form of HRF induces allergy, such as the ability to release histamine is significantly reduced compared to HRF secreted from cells. The present inventors proved for the first time that the dimer form of HRF, which solves these questions, is an allergy-inducing substance, and found a new target effective in developing a new therapeutic agent for allergy by securing the original technology (Korean Patent 10-0780255, European Patent 1683866, Japanese Patent 4564926, and U.S. Pat. No. 7,772,368).

In order to develop a therapeutic agent based on understanding the pathophysiology of this novel target dimeric HRF causing allergy, the present inventors have discovered an anti-allergic 7-mer peptide that specifically binds to dimeric HRF with a stronger affinity than to monomeric HRF and inhibits its function (Korean Patent 10-0457350, European Patent 1167526, Japanese Patent 4295449, and U.S. Pat. No. 6,710,165). In other words, the present inventors developed a peptide drug (dTBP2) with anti-allergic efficacy by targeting and controlling dimeric HRF, and demonstrated its inhibitory efficacy in allergic diseases and rheumatoid arthritis (Korean Patent 10-1830838). In addition, since the structure of HRF inducing allergy was not clearly identified, it was difficult to enter the drug development stage by screening a substance that binds to and inhibits HRF. However, our research team has elucidated the structure and identified an effective domain that is important for its function as HRF (Korean Patent 10-1830838, Korean Patent 10-1804291, Korean Patent 10-1804285, and Korean Patent 10-1843051).

On the other hand, TCTP protein (translationally controlled tumor protein) is a protein reported by MacDonald et al (1995) as an IgE-dependent histamine-releasing factor (HRF) having histamine-releasing activity. TCTP was known as a tumor-specific protein until the 1980s, and its synthesis has been thought to be related to the proliferative stage of tumor. In mouse erythroleukemia cells, it was found to be 21 kDa tumor protein p21 (Chitpatima et al, 1988), and in Ehrlich ascites tumor, the protein p23 related to cell growth was found to be the same as TCTP/HRF (Bohm et al, 1989).

Peptide drugs have the advantages of fewer side effects, fast drug effects, and biocompatibility, and 20 kinds of amino acids are easy to chemically manufacture and modify, so QC (quality control) is easy and the possibility of commercialization is high. For these reasons, peptide drugs are attracting attention worldwide in the fields of metabolic diseases (obesity, diabetes, etc.) and anticancer drugs, and are being developed as immunotherapy, hormone therapy, and rare disease therapy.

However, peptide drugs have a disadvantage of high immunogenicity when administered to a patient, and generally have low stability and are easily denatured. In addition, peptide drugs are degraded by proteolytic enzymes in the body and lose their activity, and are easily eliminated through the kidneys due to their relatively small size. Therefore, in order to maintain the blood concentration and potency of the drug containing peptides as a pharmacological component, it is necessary to frequently administer the peptide drug to the patient. However, peptide drugs are mostly limited to parenteral administration and administered to patients in the form of injections, and therefore peptide drugs are frequently injected to maintain the blood concentration of physiologically active peptides, which causes great pain to the patient. In addition, most of the peptide drugs have a problem in that the production cost is high, so that the burden on the patient is high. Various efforts have been made to overcome these problems and to maximize the drug efficacy by increasing the blood stability of the peptide drug to keep the blood drug concentration high for a long time.

A representative method for compensating for the disadvantage of peptide drugs is to use a drug delivery system. In order to select the DDS of a peptide drug, various aspects should be considered, that is, drug release rate, solubility, stability, biodegradability, toxicity, and the like should be checked. Various methods that are likely to satisfy the above factors have been continuously reported, and the most representative method among them is a method of modifying peptides.

A representative example of the methods of modifying peptides that have been currently studied is PEGylation (PEG modification technique) of a protein, in which PEG is covalently linked to a peptide or protein. PEG (polyethyleneglycol) is a representative biopolymer material approved by the FDA, and PEG having a molecular weight of about 2 kDa to 20 kDa is generally used for pegylation of proteins. PEG has the effect of reducing immunogenicity by covering the epitope on the surface of a protein recognized by an antibody, and can prevent other molecules from penetrating by absorbing water molecules in an aqueous solution. In addition, since the PEG linked to the peptide can block the access of a degrading enzyme while acting as a structural barrier (steric hindrance), the peptide drug can be retained in the body for a long time. Moreover, PEG has the effect of increasing the blood half-life of peptide drugs by reducing the rate lost by filtration in the kidneys, and various effects such as increased resistance to protein degradation and control of drug delivery and drug release rates have been reported (Inada et al. J. Bioact. and Compatible Polymers, 5, 343, 1990).

Currently, several types of PEGylated proteins are being applied clinically. In 1990, PEGylated-bovine adenosine deaminase (Adagen) produced by ENZON Inc was approved by the FDA, and is used to treat severe combined immunodeficiency diseases (pegfamg 013102LB, http://wwwfdagov). PEGylated interferon-α (PEG IFN-α2a, Pegasys) manufactured by Hoffman-la Roche Ltd was approved for sale in 2002, and is used to treat hepatitis (103964s50371b1, pegsche 011901LB, http://wwwfdagov). In 2002, PEGylated human granulocyte colony-stimulating factor (PEG-filgrastim, Neulasta) manufactured by Amgen Inc was also approved by the FDA, which is used to treat metastatic breast cancer (pegfamg 013102LB, http://wwwfdagov). As such, PEG has been demonstrated to be a safe drug modulator with a well-understood in vivo metabolism and few side effects.

As a method for modifying peptides, studies on polymers that can replace PEG are also being actively conducted. For example, poly(glycerol), poly(oxazoline), poly(vinylpyrrolidone), poly(acrylamide), poly(peptide), poly(2-alkyl-2-oxazoline), polysarcosine, poly(vinyl alcohol), polyzwitterion and the like have been reported as biocompatible polymers that can replace PEG (Srinivas Abbina et el., Engineering of Biomaterials for Drug Delivery System, 2018; Vitaliy V. Khutoryanskiy, Advanced Drug Delivery Reviews, Volume 124, 140-149).

Among them, an example of peptide polymer fusion, which is a method of modifying using polypeptide, is PASylation in which proline-alanine-serine residues are repeated. PAS is a water-insoluble and non-charged polymer and has been reported to have biophysical properties similar to those of PEG. (Martin Schlapschy et al., Protein Engineering, Design & Selection, vol. 26, no. 8, 489-501, 2013).

Another example of peptide polymer fusion is elastin-like polypeptides (ELPs). ELP is a polypeptide derived from mammalian elastin and synthesized by genetic recombination. It is a peptide in which five amino acids of valine-proline-glycine-Xaa-glycine are repeated, where Xaa consists of amino acids excluding proline. According to studies, both cationic and anionic drugs can be easily released by controlling the ionic strength of ELP. ELP has a temperature-sensitive property, so when combined with ionic amino acids such as lysine, glutamic acid and aspartic acid in the Xaa moiety, ELP has very high lower critical solution temperature (LCST). When ELP is combined with a substance having opposite ionicity, LCST is very low. Using these characteristics, studies are being conducted on a drug delivery system that is insoluble when the ELP capsule is formed together with the drug, but dissolves naturally when the drug disappears (Jae-Yeon Jeong et al., Biomaterials Research, 2008, 12, 2, 39-47). As a patent for a protein pharmaceutical formulation using ELP, an invention related to a sustained-release pharmaceutical formulation including a protein activator and an elastin-like peptide (ELP), filed by Phasebio Pharmaceuticals Inc, has been disclosed (Korean Patent KR10-2019-0005171).

Another representative example of peptide polymer fusion is XTEN technology of Amunix Pharmaceuticals Inc. XTEN, a recombinant polypeptide, provides a technology capable of producing a fusion protein through one-step expression (homogeneous product) by improving the existing technology for producing a fusion protein that goes through two or more steps (heterogeneous product). In addition, XTEN has the advantage of extending the half-life of the drug and low immunogenicity. In relation to this, it is reported that Roche recently partnered with Amunix's Xten technology for an upfront payment of $40 million to introduce a platform for extending the half-life of drugs, and agreed to apply it to undisclosed targets other than anticancer drugs and pay a development and sales milestone of $1.5 billion in the future.

Another representative example of a method being developed to improve the stability of peptide drugs and increase the half-life in the body is albumin fusion. Albumin fusion peptide technology is drawing attention as an alternative technology to PEGylation because it increases the stability of the peptide in plasma by linking the recombinant albumin to the protein with a linker, has few side effects, and has advantages in terms of price. As a representative example of albumin fusion technology, it was recently reported that the hemophilia treatment Idelvion of Australian pharmaceutical company CLS Behring using albumin fusion technology received FDA approval in 2016.

As one of the methods being developed as a means for transporting peptide drugs, there is a technology using cyclotide as a scaffold. Cyclotide is a disulfide bond rich peptide isolated from plants, typically containing 28-37 amino acids, and characterized by a head and tail cyclized peptide backbone and three disulfide bonds interlocked with each other. Cyclotide began to attract attention in that it binds to a drug and enables oral administration of the drug (Andrew Gould et al., Curr Pharm Des. 2011, 12, 17(38), 4294-4307).

In an effort to increase the stability of peptide drugs, antibody-based biopersistent drug platform technology is one of the technologies that should be noted with high bio-application potential. Representative technologies include Lapscovery (Long Acting Protein/Peptide Discovery Platform Technology, Hanmi Pharm. Co., Ltd.) and HyFc (Hybid Fc) technology (Genexine, Inc.).

Hanmi Pharm's Lapscovery technology minimizes steric hindrance by linking the drug and the polymer (IgG1 Fc) with a linker (eg, PEG). By doing this, one of the disadvantages of PEGylation, protein activity degradation due to difficulty in binding to a specific site, was overcome. In addition, there are advantages such as recyclability of the drug in vivo and persistence according to a high half-life. Labscovery technology was registered as a domestic patent in 2011 (Korean Patent KR10-2008-0064750).

Genexine's HyFC technology is a technology for a fusion protein made by fusing the strengths of IgG4 and IgD among the types of antibodies in our body, and it maximizes biological activity and persistence in the body. At this time, Ig4 plays a role of increasing the half-life, and IgD plays a role of minimizing mutual interference by maximizing the flexibility of a hinge, thereby greatly increasing drug activity. The original patent for HyFC technology was registered as a domestic patent in 2009 (Korean Patent KR10-2008-0094781). As patents for therapeutic agents in which HyFC technology is applied, a long-acting growth hormone (U.S. Pat. No. 8,586,038 B2), a therapeutic agent for anemia (U.S. Pat. No. 8,586,531 B2), and a therapeutic agent for leukopenia (U.S. Pat. No. 8,586,048 B2) were registered in the United States in 2013.

As a method to compensate for the disadvantages of peptide drugs in addition to the above-mentioned modification, fusion, and antibody-based technologies, various drug delivery methods such as a method of injecting a drug using nanoparticles (liposomes, exosomes, etc.) or coating and loading a drug on the skin through a patch or microneedle, and a drug delivery method using a cell penetrating peptide are being developed. As described above, research and development on biobetter, a drug that has improved efficacy, safety, and convenience compared to existing biopharmaceuticals, are being actively conducted.

### SUMMARY OF THE INVENTION

It is an object of the present invention to provide a modification method of a HRF-binding peptide.

It is another object of the present invention to provide a composition for preventing or treating allergy containing the modified HRF-binding peptide.

It is another object of the present invention to provide a composition for preventing or treating malaria containing the modified HRF-binding peptide.

It is another object of the present invention to provide a composition for preventing or treating autoimmune disease containing the modified HRF-binding peptide.

It is another object of the present invention to provide a composition for preventing or treating acute or chronic inflammatory disease containing the modified HRF-binding peptide.

It is another object of the present invention to provide a composition for preventing or treating hypertension containing the modified HRF-binding peptide.

It is another object of the present invention to provide a composition for preventing or treating cancer containing the modified HRF-binding peptide.

It is another object of the present invention to provide a method for treating allergy, malaria, autoimmune disease, acute or chronic inflammatory disease, hypertension or cancer, comprising a step of administering the PEGylated HRF-binding peptide to an individual or subject in need thereof.

It is another object of the present invention to provide the PEGylated HRF binding peptide for use in the treatment of allergy, malaria, autoimmune disease, acute or chronic inflammatory disease, hypertension or cancer.

It is another object of the present invention to provide a use of the PEGylated HRF-binding peptide for the preparation of a medicament for the treatment of allergy, malaria, autoimmune disease, acute or chronic inflammatory disease, hypertension or cancer.

To achieve the above objects, the present invention provides a PEGylated HRF-binding peptide in which polyethylene glycol is bound to the HRF peptide comprising a sequence of amino acids wherein the first amino acid is selected from the group consisting of A, L and W; the second amino acid is selected from the group consisting of V, Y, E and A; the third amino acid is selected from the group consisting of T, V, F and A; the fourth amino acid is selected from the group consisting of Y, P and A; the fifth amino acid is selected from the group consisting of P, G and K; the sixth amino acid is selected from the group consisting of A, L, S and W; and the seventh amino acid consists of a sequence of amino acids selected from the group consisting of A, P and M.

The present invention also provides a composition for preventing or treating allergy containing the PEGylated HRF-binding peptide.

The present invention also provides a composition for preventing or treating malaria containing the PEGylated HRF-binding peptide.

The present invention also provides a composition for preventing or treating autoimmune disease containing the PEGylated HRF-binding peptide.

The present invention also provides a composition for preventing or treating acute or chronic inflammatory disease containing the PEGylated HRF-binding peptide.

The present invention also provides a composition for preventing or treating hypertension containing the PEGylated HRF-binding peptide.

The present invention also provides a composition for preventing or treating cancer containing the PEGylated HRF-binding peptide.

The present invention also provides a method for treating allergy, malaria, autoimmune disease, acute or chronic inflammatory disease, hypertension or cancer, comprising a step of administering the PEGylated HRF-binding peptide to an individual or subject in need thereof.

The present invention also provides a PEGylated HRF-binding peptide for use in the treatment of allergy, malaria, autoimmune disease, acute or chronic inflammatory disease, hypertension or cancer. In addition, the present invention provides a use of the PEGylated HRF-binding peptide for the preparation of a medicament for the treatment of allergy, malaria, autoimmune disease, acute or chronic inflammatory disease, hypertension or cancer.

### Advantageous Effect

The modified HRF-binding peptide according to the present invention can exert drug efficacy with high stability in vivo, can be effectively used as a drug because the number of administrations can be reduced due to its longer half-life, and can prevent or treat allergies, malaria, autoimmune diseases, acute or chronic inflammatory diseases, hypertension, and cancer in humans as well as animals by effectively inhibiting histamine secretion in cells.

### BRIEF DESCRIPTION OF THE DRAWINGS

FIG. 1 is a graph showing the HPLC results of the PEGylated HRF-binding peptide of Example 1 according to the present invention.

FIG. 2 is a diagram showing the MALDI-TOF mass spectra of the compounds of Preparative Example 1 (top) and Example 1 (bottom) according to the present invention.

FIG. 3 is a graph showing the HPLC results of the PEGylated HRF-binding peptide of Example 2 according to the present invention.

FIG. 4 is a diagram showing the MALDI-TOF mass spectra of the compounds of Preparative Example 2 (top) and Example 2 (bottom) according to the present invention.

FIG. 5 is a graph showing the HPLC results of the PEGylated HRF-binding peptide of Example 3 according to the present invention.

FIG. 6 is a diagram showing the MALDI-TOF mass spectrum of the compound of Example 3 according to the present invention.

FIG. 7 is a diagram showing the experimental results for the inhibitory effect of the compounds of Preparative Example 4, Example 1 and Example 2 according to the present invention by HRF on IL-8 secretion in BEAS-2B cells.

FIG. 8 is a diagram showing the simple schematic diagram of an experiment of Experimental Example 4 according to the present invention.

FIG. 9 is a diagram showing the experimental results for the inhibitory effect of the normal control (NC), positive control (PC), dexamethasone, and the compounds of Preparative Example 4 and Example 3 according to the present invention on eosinophil increase.

FIG. 10 is a diagram showing the experimental results for the inhibitory effect of the normal control (NC), positive control (PC), and the compounds of Preparative Example 4 and Example 3 on IL-5 secretion (administration of the compounds of Preparative Example 4 and Example 3 once a day for a total of 8 times).

FIG. 11 is a diagram showing the experimental results for the inhibitory effect of the normal control (NC), positive control (PC), and the compounds of Preparative Example 4 and Example 3 on IL-5 secretion (administration of the compounds of Preparative Example 4 and Example 3 once in 4 days for a total of 2 times).

FIG. 12 is a diagram showing the experimental results for the inhibitory effect of the normal control (NC), positive control (PC), and the compounds of Preparative Example 4 and Example 3 on IL-5 secretion (administration of the compounds of Preparative Example 4 and Example 3 once on the 1^{st} day of 8 days inducing bronchial asthma and rhinitis).

FIG. 13 is a diagram showing the experimental results for the inhibitory effect of the normal control (NC), positive control (PC), and the compounds of Preparative Example 4 and Example 3 on IL-5 secretion (administration of the compounds of Preparative Example 4 and Example 3 once on the 5^{th} day of 8 days inducing bronchial asthma and rhinitis).

FIG. 14 is a diagram showing the experimental results for the inhibitory effect of the normal control (NC), positive control (PC), and the compounds of Preparative Example 4 and Example 3 on IL-4 secretion (administration of the compounds of Preparative Example 4 and Example 3 once a day for a total of 8 times).

FIG. 15 is a diagram showing the experimental results for the inhibitory effect of the normal control (NC), positive control (PC), and the compounds of Preparative Example 4 and Example 3 on IL-4 secretion (administration of the compounds of Preparative Example 4 and Example 3 once in 4 days for a total of 2 times).

FIG. 16 is a diagram showing the experimental results for the inhibitory effect of the normal control (NC), positive control (PC), and the compounds of Preparative Example 4 and Example 3 on IL-4 secretion (administration of the compounds of Preparative Example 4 and Example 3 once on the 1^{st} day of 8 days inducing bronchial asthma and rhinitis).

FIG. 17 is a diagram showing the experimental results for the inhibitory effect of the normal control (NC), positive control (PC), and the compounds of Preparative Example 4 and Example 3 on IL-4 secretion (administration of the compounds of Preparative Example 4 and Example 3 once on the 5^{th} day of 8 days inducing bronchial asthma and rhinitis).

FIG. 18 is a diagram showing the experimental results for the inhibitory effect of the normal control (NC), positive control (PC), and the compounds of Preparative Example 4 and Example 3 on IL-13 secretion (administration of the compounds of Preparative Example 4 and Example 3 once a day for a total of 8 times).

FIG. 19 is a diagram showing the experimental results for the inhibitory effect of the normal control (NC), positive control (PC), and the compounds of Preparative Example 4 and Example 3 on IL-13 secretion (administration of the compounds of Preparative Example 4 and Example 3 once in 4 days for a total of 2 times).

FIG. 20 is a diagram showing the experimental results for the inhibitory effect of the normal control (NC), positive control (PC), and the compounds of Preparative Example 4 and Example 3 on IL-13 secretion (administration of the compounds of Preparative Example 4 and Example 3 once on the 1^{st} day of 8 days inducing bronchial asthma and rhinitis).

FIG. 21 is a diagram showing the experimental results for the inhibitory effect of the normal control (NC), positive control (PC), and the compounds of Preparative Example 4 and Example 3 on IL-13 secretion (administration of the compounds of Preparative Example 4 and Example 3 once on the 5^{th} day of 8 days inducing bronchial asthma and rhinitis).

FIG. 22 is a diagram showing the experimental results for the inhibitory effect of the normal control (NC), positive control (PC), and the compounds of Preparative Example 4 and Example 3 on ovalbumin-specific IgE secretion (administration of the compounds of Preparative Example 4 and Example 3 once a day for a total of 8 times).

FIG. 23 is a diagram showing the experimental results for the inhibitory effect of the normal control (NC), positive control (PC), and the compounds of Preparative Example 4 and Example 3 on ovalbumin-specific IgE secretion (administration of the compounds of Preparative Example 4 and Example 3 once in 4 days for a total of 2 times).

FIG. 24 is a diagram showing the experimental results for the inhibitory effect of the normal control (NC), positive control (PC), and the compounds of Preparative Example 4 and Example 3 on ovalbumin-specific IgE secretion (administration of the compounds of Preparative Example 4 and Example 3 once on the 1^{st} day of 8 days inducing bronchial asthma and rhinitis).

FIG. 25 is a diagram showing the experimental results for the inhibitory effect of the normal control (NC), positive control (PC), and the compounds of Preparative Example 4 and Example 3 on ovalbumin-specific IgE secretion (administration of the compounds of Preparative Example 4 and Example 3 once on the 5^{th} day of 8 days inducing bronchial asthma and rhinitis).

FIG. 26 is a diagram showing the results of confirming the concentration changes of the compounds of Preparative Example 4 and Example 3 in male mice over time.

### DESCRIPTION OF THE PREFERRED EMBODIMENTS

Hereinafter, the present invention is described in detail.

The present invention provides a PEGylated HRF-binding peptide in which polyethylene glycol is bound to the HRF peptide comprising a sequence of amino acids wherein the first amino acid is selected from the group consisting of A, L and W; the second amino acid is selected from the group consisting of V, Y, E and A; the third amino acid is selected from the group consisting of T, V, F and A; the fourth amino acid is selected from the group consisting of Y, P and A; the fifth amino acid is selected from the group consisting of P, G and K; the sixth amino acid is selected from the group consisting of A, L, S and W; and the seventh amino acid consists of a sequence of amino acids selected from the group consisting of A, P and M.

The molecular weight of the polyethylene glycol is not limited as long as it is a molecular weight capable of exhibiting the activity of the HRF-binding peptide according to the present invention, but can be 1 kDa to 50 kDa, 2 kDa to 45 kDa, 3 kDa to 40 kDa, 4 kDa to 35 kDa, 5 kDa to 30 kDa, 3 kDa to 25 kDa, 3 kDa to 20 kDa, 3 kDa to 15 kDa, 3 kDa to 13 kDa, 4 kDa to 6 kDa, 5 kDa, 5 kDa to 10 kDa, 9 kDa to 11 kDa, or 10 kDa.

The binding of the HRF-binding peptide to polyethylene is due to the covalent bond of polyethylene glycol to the carboxyl group or amino group of the HRF-binding peptide.

The polyethylene glycol can be connected with a functional group including an aldehyde group, a carboxyl group, an amino group, or a hydrazide group at the terminal.

The functional group can be connected to the terminal oxygen of polyethylene glycol through C₁₋₆ alkylene.

The other terminal of the polyethylene glycol can be capped. At this time, the terminal can be capped with an alkoxy group, for example, can be capped with a methoxy group.

The polyethylene glycol may be methoxy polyethylene glycol propion aldehyde (5000), methoxy polyethylene glycol hydrazide (5000), or methoxy polyethylene glycol propion aldehyde (1000).

The polyethylene glycol or a derivative thereof can be bound to the N-terminal or C-terminal of the peptide.

The HRF-binding peptide can be composed of a sequence of amino acids in which the first amino acid is selected from A or W; the second amino acid is selected from Y or A; the third amino acid is selected from V or A; the fourth amino acid is selected from Y or A; the fifth amino acid is selected from P or K; the sixth amino acid is selected from S or A; and the seventh amino acid is selected from M or A.

Or, the HRF-binding peptide can be composed of a sequence of amino acids in which the first amino acid is W; and the seventh amino acid is M.

Preferably, the HRF-binding peptide can be composed of the amino acid sequence W-Y-V-Y-P-S-M; A-Y-V-Y-P-S-M; or W-Y-V-A-P-S-M.

Most preferably, the HRF-binding peptide can be composed of the amino acid sequence W-Y-V-Y-P-S-M.

The HRF-binding peptide can be a peptide consisting of L-, D-, or L- and D-amino acids.

The HRF-binding peptide can be a peptide comprising one or more modified amino acids.

The modified amino acid can be an amino acid derivative or an alkylated amino acid.

The HRF-binding peptide has an effect of inhibiting IL-8 secretion.

The HRF-binding peptide has an effect of inhibiting eosinophil increase.

The HRF-binding peptide has an effect of inhibiting IL-5 secretion.

The HRF-binding peptide has an effect of inhibiting IL-4 secretion.

The HRF-binding peptide has an effect of inhibiting IL-13 secretion.

The HRF-binding peptide has an effect of inhibiting ovalbumin-specific IgE secretion.

The peptide comprising the amino acid sequence W-Y-V-Y-P-S-M is a heptamer named dTBP2 [dTCTP (dimerized translationally controlled tumor protein) binding peptide 2] prepared by the present inventors (Korean Laid-Open Patent Publication No. 10-2017-0004906), and was confirmed by inhibiting the action of dTCTP. The chemical structure of dTBP2 is as follows.

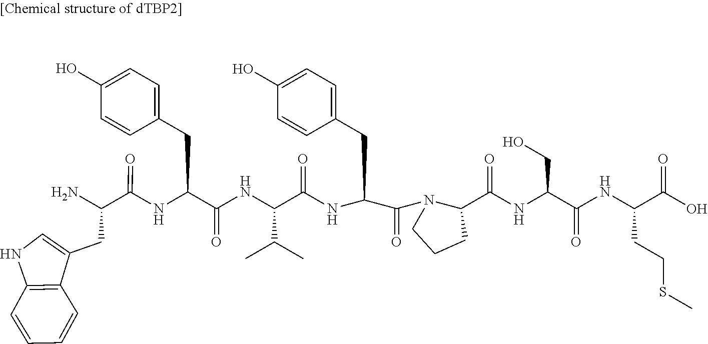 [SMILES: CSCC[C@H](NC(=O)[C@H](CO)NC(=O)[C@@H]1CCCN1C(=O)[C@H](Cc1ccc(O)cc1)NC(=O)[C@@H](NC(=O)[C@H](Cc1ccc(O)cc1)NC(=O)[C@@H](N)Cc1c[nH]c2ccccc12)C(C)C)C(=O)O]

As described in Korean Laid-Open Patent Publication No. 10-2017-0004906, it was confirmed that the activity of dTBP2 was equally maintained even if the first amino acid tryptophan and the fourth amino acid tyrosine were substituted with alanine.

The present invention provides a composition for preventing or treating allergy containing the PEGylated HRF-binding peptide. The allergy can be asthma, rhinitis, atopy, urticaria, anaphylaxis, allergic bronchiectasis, allergy caused by food, drug, pollen, or insects, allergic conjunctivitis, hay fever, cold urticaria, or atopic dermatitis.

The present invention provides a composition for preventing or treating malaria containing the PEGylated HRF-binding peptide. The malaria can be caused by Plasmodium falciparum, Plasmodium vivax, Plasmodium ovale, or Plasmodium malariae.

The present invention provides a composition for preventing or treating autoimmune disease containing the PEGylated HRF-binding peptide. The autoimmune disease can be rheumatoid arthritis, Sjogrean's disease, systemic sclerosis, polymyositis, systemic angitis, mixed connective tissue disease, Crohn's disease, Hashimoto's disease, Grave's disease, Goodpasture's syndrome, Guillain-Barre syndrome, idiopathic thrombocytopenic purpura, irritable bowel syndrome, myasthenia gravis, narcolepsy, pemphigus vulgaris, pernicious anemia, primary biliary cirrhosis, ulcerative colitis, vasculitis, Wegener's granulomatosis, psoriasis, alopecia areata, rheumatic fever, systemic lupus erythematosus, or multiple scleorosis.

The present invention provides a composition for preventing or treating acute or chronic inflammatory disease containing the PEGylated HRF-binding peptide. The acute or chronic inflammatory disease can be conjunctivitis, periodontitis, rhinitis, otitis media, pharyngitis, tonsillitis, dermatitis, gastritis, colitis, ankylosing spondylitis, psoriatic arthritis, osteoarthritis, rheumatoid arthritis, periarthritis, tendinitis, xerosis, periostitis, myositis, hepatitis, cystitis, nephritis, pneumonia, gastric ulcer, Crohn's disease, Sjogrean's disease, gout, fibromyalgia, lupus, bursitis, or systemic lupus erythematodes.

The present invention provides a composition for preventing or treating hypertension containing the PEGylated HRF-binding peptide.

The present invention provides a composition for preventing or treating cancer containing the PEGylated HRF-binding peptide. The cancer can be oral cancer, liver cancer, stomach cancer, colon cancer, breast cancer, lung cancer, bone cancer, pancreatic cancer, skin cancer, head and neck cancer, skin cancer, cervical cancer, ovarian cancer, colorectal cancer, small intestine cancer, rectal cancer, fallopian tube carcinoma, perianal cancer, endometrial carcinoma, vaginal carcinoma, vulvar carcinoma, Hodgkin's disease, esophageal cancer, lymph adenocarcinoma, bladder cancer, gallbladder cancer, endocrine adenocarcinoma, thyroid cancer, parathyroid cancer, adrenal cancer, soft tissue sarcoma, urethral cancer, penile cancer, prostate cancer, chronic leukemia, acute leukemia, lymphocytic lymphoma, renal cancer, ureter cancer, renal cell carcinoma, renal pelvic carcinoma, central nervous system tumor, primary central nervous system lymphoma, spinal cord tumor, brainstem glioma or pituitary adenoma.

In addition to the method of modification by pegylating the HRF-binding peptide with polyethylene glycol or its derivative as described above, a technique using cyclotide or cyclic peptide as a scaffold can be applied to the HRF-binding peptide according to the present invention.

That is, in one aspect of the present invention, there is provided a conjugate peptide in which the HRF-binding peptide and cyclotide are combined, the cyclotide contains 25 to 35 amino acids, and a head and a tail are cyclized.

The cyclotide contains three disulfide bonds.

The three disulfide bonds may be interlocking.

The cyclotide may be derived from a plant.

The plant can be at least one selected from the group consisting of C. parcifolia, P. longipes, V. odorata, O. affinis, P. condensata, V. tricolor, V. arvensis and M. cochinchinensis.

The conjugate may be an oral formulation.

In addition to the method of modification by pegylating the HRF-binding peptide according to the present invention with polyethylene glycol or its derivative as described above, the following compounds can be used instead of polyethylene glycol.

For example, biocompatible polymers such as poly(glycerol), poly(oxazoline), poly(vinylpyrrolidone), poly(acrylamide), poly(peptide), poly(2-alkyl-2-oxazoline), polysarcosine, poly(vinyl alcohol), polyzwitterion, and the like can be bound to the HRF-binding peptide according to the present invention directly or via a linker. However, as a method for modifying the HRF-binding peptide according to the present invention, the compound capable of replacing polyethylene glycol is not limited to the above, and as a compound having biocompatibility, it is not limited as long as it is a compound that can compensate for the disadvantages of a peptide drug.

That is, in one aspect of the present invention, there is provided an HRF-binding peptide modified with one compound selected from the group consisting of poly(glycerol), poly(oxazoline), poly(vinylpyrrolidone), poly(acrylamide), poly(peptide), poly(2-alkyl-2-oxazoline), polysarcosine, poly(vinyl alcohol) and polyzwitterion.

In addition, the HRF-binding peptide according to the present invention can be modified using a polypeptide, which is one of the polymers that can replace the polyethylene glycol. For example, the HRF-binding peptide according to the present invention can be linked (bound) to the peptide in which proline-alanine-serine residues are repeated (PASylation). In this case, the PASylated HRF-binding peptide has similar properties to the PEGylated HRF-binding peptide according to the present invention.

That is, in one aspect of the present invention, there is provided an HRF-binding peptide to which the peptide having a repeating amino acid sequence P-A-S is bound or linked (PASylation).

In addition, as another example in which the HRF-binding peptide according to the present invention can be modified using a polypeptide, ELPs (elastin like polypeptides) can be bound to the HRF-binding peptide according to the present invention. ELPs are peptides in which valine-proline-glycine-Xaa-glycine residues are repeated (In this case, Xaa is an amino acid except for proline (P)).

That is, in one aspect of the present invention, there is provided a composition for diagnosing, preventing or treating allergy, malaria, autoimmune disease, acute or chronic inflammatory disease, hypertension, or cancer, containing the HRF-binding peptide and at least 60 elastin-like peptide (ELP) structural units selected from the group consisting of the sequences represented by SEQ. ID. NO: 1 to NO: 13 disclosed in Korean Laid-Open Patent Publication No. 10-2019-0005171.

In another aspect, there is provided a sustained release pharmaceutical formulation comprising the composition.

The formulation provides slow absorption from the site of injection upon administration.

The formulation provides a flat PK profile upon administration when compared to the pharmacokinetic (PK) profile of the HRF-binding peptide in the absence of the elastin-like peptide.

(Korean Laid-Open Patent Publication No. 10-2019-0005171)

In addition, as another example in which the HRF-binding peptide according to the present invention can be modified using a polypeptide, the HRF-binding peptide can be modified through XTEN technology. XTEN technology, which is a technology of Amunix, has the effect of extending the half-life of drugs in the body and has the advantage of low immunogenicity.

That is, in one aspect of the present invention, there is provided a recombinant fusion protein comprising the HRF-binding peptide and an extended recombinant polypeptide (XTEN), wherein the fusion protein exhibits an apparent molecular weight coefficient of at least about 4 and exhibits an effect of diagnosing, preventing or treating allergy, malaria, autoimmune disease, acute or chronic inflammatory disease, hypertension, cancer when administered to a subject by using a therapeutically effective amount. In this case, the XTEN has the following characteristics.
(a) XTEN contains at least 36 amino acid residues;
(b) the sum of glycine (G), alanine (A), serine (S), threonine (T), glutamate (E) and proline (P) residues account for greater than about 80% of the total amino acid residues of XTEN;
(c) (i) XTEN does not contain three contiguous amino acids that are identical unless the amino acid is serine, (ii) at least about 80% of the XTEN sequence consists of non-overlapping sequence motifs comprising each of about 9 to about 14 amino acid residues composed of 4 to 6 amino acids selected from glycine (G), alanine (A), serine (S), threonine (T), glutamate (E) and proline (P), wherein no two contiguous amino acid residues occur more than twice in each non-overlapping sequence motif, or (iii) XTEN is substantially non-repeatable such that the XTEN sequence has a subsequence score of less than 10;
(d) XTEN has greater than 90% random coil formation when measured by GOR algorithm;
(e) XTEN has less than 2% alpha helices and 2% beta-sheets when measured by Chou-Fasman algorithm; and
(f) XTEN lacks the predicted T cell epitope when analyzed by the TEPITOPE algorithm, with the TEPITOPE threshold score of −9 for that prediction by the algorithm.

(Korean Laid-Open Patent Publication No. 10-2014-0069131, Korean Laid-Open Patent Publication No. 10-2011-01276969)

In addition, the HRF-binding peptide according to the present invention can be modified using albumin fusion technology. That is, the recombinant albumin can be linked to the HRF-binding peptide.

That is, in one aspect of the present invention, there is provided an albumin-fused HRF-binding peptide in which the HRF-binding peptide and the recombinant albumin are linked by a cleavable peptide linker.

In addition, antibody-based biopersistence drug platform technology can be applied to the HRF-binding peptide according to the present invention. That is, lapscovery (Long Acting Protein/Peptide Discovery Platform Technology, Hanmi Pharm. Co., Ltd.) or HyFc (Hybid Fc) technology (Genexine, Inc.) can be applied to the HRF-binding peptide according to the present invention.

That is, in one aspect of the present invention, there is provided an HRF-binding peptide conjugate in which the HRF-binding peptide and an immunoglobulin Fc region are linked via a non-peptidyl polymer selected from the group consisting of polyethylene glycol, polypropylene glycol, ethylene glycol-propylene glycol copolymer, polyoxyethylated polyol, polyvinyl alcohol, polysaccharide, dextran, polyvinyl ethyl ether, biodegradable polymer, lipid polymer, chitin, hyaluronic acid and a combination thereof.

(Korean Laid-Open Patent Publication No. 10-2008-0064750)

That is, in one aspect of the present invention, there is provided a conjugate in which the HRF-binding peptide is bound to a hybrid Fc, wherein the hybrid Fc is derived from a combination of human IgG subclass or a combination of human IgD and IgG.

Specifically, it is a hybrid human immunoglobulin Fc fragment comprising a hinge region, a CH2 domain and a CH3 domain in the N-terminal to C-terminal direction. The hinge region is at least a partial amino acid sequence of a human IgD hinge region or a human IgG1 hinge region. The CH2 domain is a human IgG4 CH2 domain in which the N-terminal region is substituted with 4-37 amino acid residues of the N-terminal region of a human IgG2 CH2 or human IgD CH2 domain.

The conjugate is a conjugate in which the HRF-binding peptide is linked to the polypeptide represented by the following formula: (Korean Laid-Open Patent Publication No. 10-2008-0094781)

N′-(Z1)p-Y-Z2-Z3-Z4-C′

Wherein, N′ is the N-terminus of the polypeptide and C′ is the C-terminus of the polypeptide;

p is an integer of 0 or 1; and

(i) Z1 is an amino acid sequence having the amino acid residues at positions 90 to 98 of SEQ. ID. NO: 11,

Y is an amino acid sequence having the amino acid residues at positions 99 to 113 of SEQ. ID. NO: 11.

Z2 is an amino acid sequence having the amino acid residues at positions 111 to 116 of SEQ. ID. NO: 12,

Z3-Z4 is an amino acid sequence consisting of continuous amino acid sequences having the amino acid residues at positions 118 to 220 of SEQ. ID. NO: 13 and the amino acid residues at positions 221 to 327 of SEQ. ID. NO: 13, or

(ii) Z1 is an amino acid sequence having the amino acid residues at positions 90 to 98 of SEQ. ID. NO: 14,

Y is an amino acid sequence having the amino acid residues at positions 158 to 162 of SEQ. ID. NO: 14, the amino acid residues at positions 153 to 162 of SEQ. ID. NO: 14, the amino acid residues at positions 148 to 162 of SEQ. ID. NO: 14, the amino acid residues at positions 143 to 162 of SEQ. ID. NO: 14, the amino acid residues at positions 133 to 162 of SEQ. ID. NO: 14, or the amino acid residues at positions 99 to 162 of SEQ. ID. NO: 14,

Z2 is an amino acid sequence having the amino acid residues at positions 163 to 170 of SEQ. ID. NO: 14,

Z3-Z4 is an amino acid sequence consisting of continuous amino acid sequences having the amino acid residues at positions 121 to 220 of SEQ. ID. NO: 13 and the amino acid residues at positions 221 to 327 of SEQ. ID. NO: 13.

The HRF-binding peptide can be fused to the N-terminus or C-terminus of the hybrid Fc, and the HRF-binding peptide may exhibit an increased circulating half-life compared to the circulating half-life of the native form of the HRF-binding peptide.

The HRF-binding peptide and the hybrid Fc can be linked by a linker.

The linker can be an albumin linker or a synthetic linker,

The albumin linker may include the residues at positions 321 to 323, 318 to 325, 316 to 328, 313 to 330, 311 to 333, or 306 to 338 of the sequence represented by SEQ. ID. NO: 25 disclosed in Korean Laid-Open Patent Publication No. 10-2008-0094781.

The synthetic linker can be a peptide of 10 to 20 amino acid residues composed of Gly and Ser residues.

The synthetic linker can be GGGGSGGGGSGGGSG.

(Korean Laid-Open Patent Publication No. 10-2008-0094781)

In addition, the HRF-binding peptide according to the present invention can be administered by using nanoparticles (liposomes, exosomes, etc.), or by coating or loading on the skin through a patch or microneedle.

In addition, the HRF-binding peptide according to the present invention can be used for drug delivery using a cell-penetrating peptide. For example, the peptide disclosed in Korean Laid-Open Patent Publication No. 10-2017-0114997 can be used.

That is, in one aspect of the present invention, there is provided a composition for diagnosing, preventing or treating allergy, malaria, autoimmune disease, acute or chronic inflammatory disease, hypertension or cancer, containing the HRF-binding peptide and the peptide consisting of the following amino acid sequence:

R1-R2-R3-R4-R5-R6-R7-R8-R9-R10
R1 is any one amino acid selected from M, L or P,
R2 is any one amino acid selected from I, A, L, P or H,
R3 is any one amino acid selected from I, L, A, P, or H,
R4 is any one amino acid selected from F, E, A, L, P or H,
R5 is any one amino acid selected from R or K,
R6 is any one amino acid selected from A, M, I, P, H or L,
R7 is any one amino acid selected from L, A, P or H,
R8 is any one amino acid selected from I, L, A, P or H,
R9 is any one amino acid selected from S, E or Y,
R10 is any one amino acid selected from H, K, R, P or L, and any one amino acid selected from KK, KKK, and KKKK can be added thereto. However, except when the sequence is MIIFRIAASHKK, MIIFRALISHKK, MIIFRAAASHKK, LIIFRIAASHKK, MIIFRIAAYHKK, MIIFKIAASHKK, LIIFRILISHKK, or MIIFRILISHKK.

Meanwhile, the amino acid sequence of the peptide according to the present invention can be modified according to the conventional techniques known in the art. For example, the peptide of the present invention can be modified by increasing or decreasing the number of amino acids. In addition, within a range that does not reduce the activity of the peptide according to the present invention, the peptide can be modified by changing the order of specific residue components except for the residues that are directly involved in binding or must be conserved. Modifiable amino acids can be modified not only with natural L-α-amino acids, but also with β, γ, δ amino acids as well as D-α-amino acid derivatives. Typically, as a result of examining the effect of electrostatic force or hydrophilicity on binding using a peptide in which one amino acid is substituted, it can be seen that the sensitivity changes when positively charged amino acids (for example, Lys, Arg, His) or negatively charged amino acids (for example, Glu, Asp, Asn, Gln) are substituted. As such, the number or type of the residues to be substituted or added is determined by the required space between the essential binding points and the required functions such as hydrophilicity or hydrophobicity. By such substitution, the affinity of the peptide according to the present invention to the target protein can be further increased. Significant changes in function can also result from the substitution. The selection of the residue to be modified has a great influence on the electrical conductivity, hydrophobicity and maintaining the basic backbone of the peptide such as side chain change or helix structure change of the molecule present at the target position. In general, substitution of a hydrophilic residue such as serine with a hydrophobic residue such as leucine, isoleucine, phenylalanine, valine or alanine, substitution of an electrically positive residue such as lysine, arginine or histidine with an electrically negative residue such as glutamic acid or aspartic acid, or substitution of an amino acid having no side chain, such as glycine, with a residue having a bulky side chain causes a significant change in the properties of a peptide. In consideration of the above-mentioned facts, those of ordinary skill in the art can modify the specific peptide using conventional techniques within the range of maintaining, or not increasing/impairing its binding ability to HRF and the histamine secretion inhibitory activity, and this is within the scope of the present invention.

Hereinafter, the present invention will be described in detail by the following examples and experimental examples. However, the following examples and experimental examples are only for illustrating the present invention, and the contents of the present invention are not limited thereto.

### <Preparative Example 1> Preparation of Methoxy Polyethylene Glycol Propionaldehyde (5000) (aldehyde-PEG (5000))

Methoxy polyethylene glycol propionaldehyde (5 kDa) was purchased from NOF CORPORATION (Japan) and prepared.

### <Preparative Example 2> Preparation of Methoxy Polyethylene Glycol Hydrazide (5000) (hydrazide-PEG (5000))

Methoxy polyethylene glycol hydrazide (5 kDa) was purchased from SunBio (Korea) and prepared.

### <Preparative Example 3> Preparation of Methoxy Polyethylene Glycol Propionaldehyde (10000) (aldehyde-PEG (10000))

Methoxy polyethylene glycol propionaldehyde (10 kDa) was purchased from NOF CORPORATION (Japan) and prepared.

### Preparative Example 4

A peptide having an amino acid sequence of WYVYPSM (dTBP2) was prepared according to Example 1 of Korean Laid-Open Patent Publication No. 10-2017-0004906.

### <Example 1> Preparation of N-Terminal 5K PEG-Conjugated dTBP2 (aldehyde-PEG (5000)-dTBP2)

The N-terminal 5K PEG-conjugated dTBP2 (aldehyde-PEG (5000)-dTBP2) prepared by the method described below was supplied and used. Specifically, 5 mg of dTBP2 prepared in Preparative Example 4 was dissolved in purified water to prepare a peptide solution at a concentration of 10 mg/mL, and 53 mg of methoxy polyethylene glycol propionaldehyde prepared in Preparative Example 1 was dissolved in 0.1 M acetate buffer (40 mM NaCNBH₃ (pH 5.5)) to prepare a PEG solution. After mixing the solution of Preparative Example 4 and the solution of Preparative Example 1 (reaction molar ratio of 1:2), the mixture was reacted at 4° C. for 18 hours to prepare the N-terminal 5K PEG-conjugated dTBP2 of Example 1.

### <Example 2> Preparation of C-Terminal 5K PEG-Conjugated dTBP2 (hydrazide-PEG (5000)-dTBP2)

The C-terminal 5K PEG-conjugated dTBP2 (hydrazide-PEG (5000)-dTBP2) prepared by the method described below was supplied and used. Specifically, 2 mg of dTBP2 prepared in Preparative Example 4 was dissolved in purified water to prepare a peptide solution at a concentration of 5 mg/mL, and 52.2 mg of methoxy polyethylene hydrazide prepared in Preparative Example 2 was dissolved in 50 mM MES buffer (pH 4.4) to prepare a PEG solution. Then, the solution of Preparative Example 4 and the solution of Preparative Example 2 were added (reaction molar ratio of 1:5) to the dTBP2 peptide solution, followed by stirring at room temperature for 10 minutes. The C-terminal 5K PEG-conjugated dTBP2 according to Example 2 was prepared by reacting 4 mg of EDAC (N-(3-methylaminopropyl)-N-ethylcarbodiimide hydrochloride) at room temperature for 1 hour and 30 minutes.

### <Example 3> Preparation of N-Terminal 10K PEG-Conjugated dTBP2 (aldehyde-PEG (10000)-dTBP2)

The N-terminal 10K PEG-conjugated dTBP2 (aldehyde-PEG (10000)-dTBP2) prepared by the method described below was supplied and used.

Specifically, 50 mg of dTBP2 prepared in Preparative Example 4 was dissolved in purified water to prepare a peptide solution at a concentration of 10 mg/mL, and 1060 mg of methoxy polyethylene hydrazide prepared in Preparative Example 3 was dissolved in 0.1 M acetate buffer (40 mM NaCNBH₃ (pH 5.5)) to prepare a PEG solution. After mixing the solution of Preparative Example 4 and the solution of Preparative Example 3 (reaction molar ratio of 1:2), the mixture was reacted at 4° C. for 18 hours.

### <Experimental Example 1> Separation and Purification of PEG (5000)-dTBP2 According to Examples 1 and 2, HPLC Analysis, and Mass Spectrometry

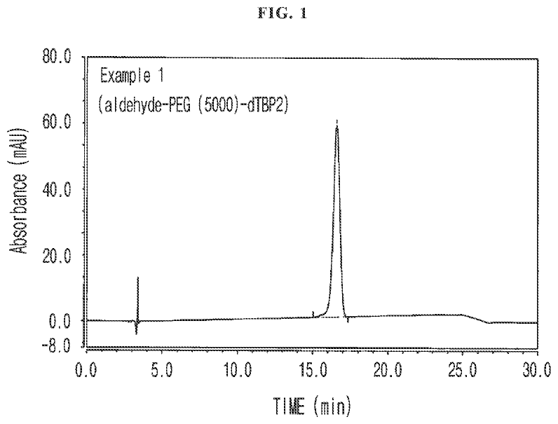

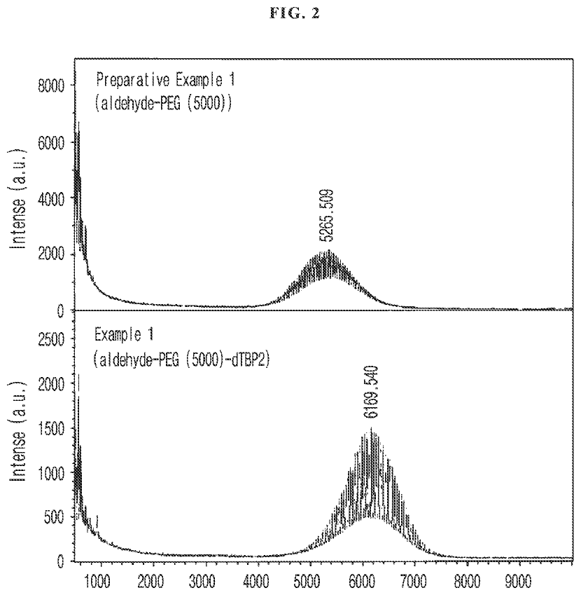

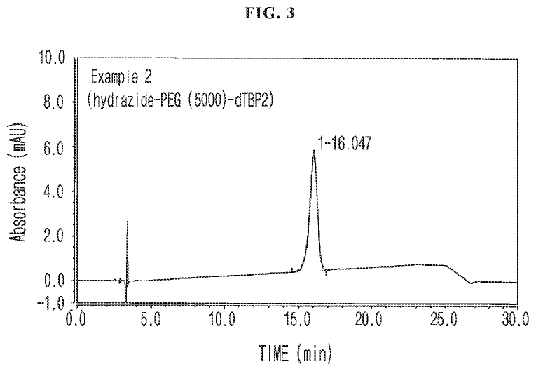

The PEG (5000)-dTBP2 prepared in Examples 1 and 2 above was dialyzed in 10 mM Tris buffer using Centricon-30, and each of the PEG (5000)-dTBP2 prepared in Examples 1 and 2 was separated using an anion exchange resin (mono-Q; Pharmacia, Sweden). The sodium salt used in the separation process was used in a concentration gradient from 0 to 300 mM. The amount of the PEG (5000)-dTBP2 according to Examples 1 and 2 separated was confirmed by size-exclusion HPLC (High Performance Liquid Chromatography) and MALDI-TOF mass spectrometry. The results are shown in FIGS. 1 to 4.

### <Experimental Example 2> Separation and Purification of PEG (10000)-dTBP2 According to Example 3, HPLC Analysis, and Mass Spectrometry

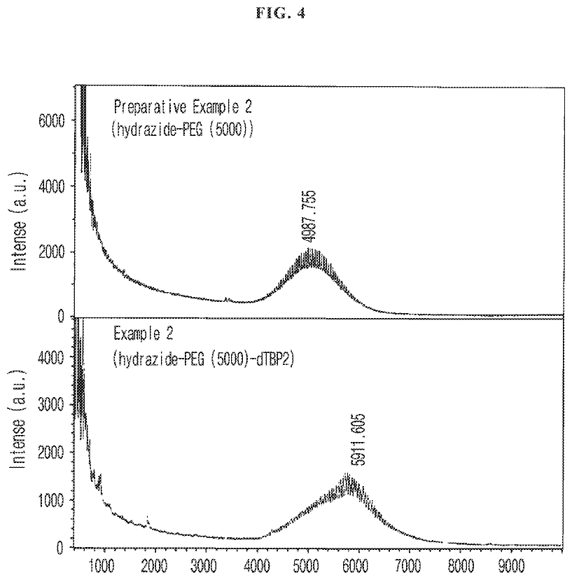

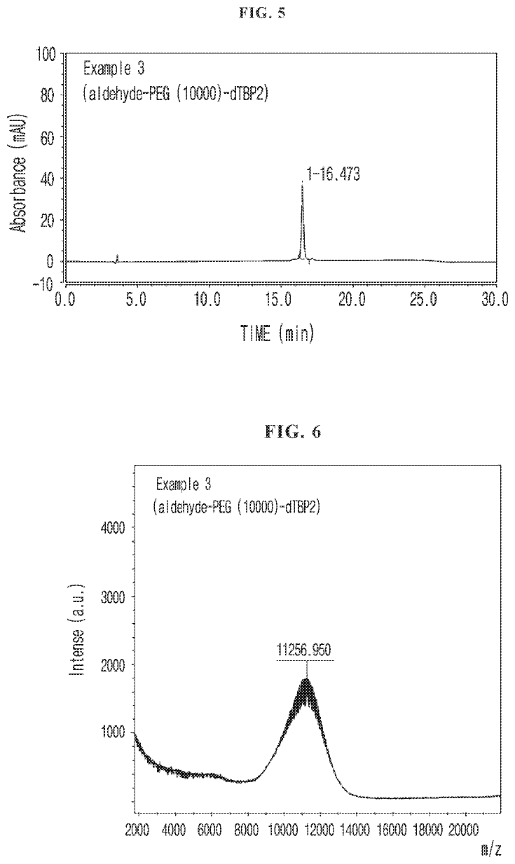

The PEG (10000)-dTBP2 prepared in Example 3 above was dialyzed in 10 mM Tris buffer using Centricon-30, and the aldehyde-PEG (10000)-dTBP2 prepared in Example 3 was separated using an anion exchange resin (mono-Q; Pharmacia, Sweden). The sodium salt used in the separation process was used in a concentration gradient from 0 to 300 mM. The amount of the aldehyde-PEG (10000)-dTBP2 according to Example 3 separated was confirmed by size-exclusion HPLC (High Performance Liquid Chromatography) and MALDI-TOF mass spectrometry. The results are shown in FIGS. 5 and 6.

### <Experimental Example 3> Confirmation of Inhibitory Effect of dTBP2 and PEG (5000)-dTBP2 According to Examples 1 and 2 on IL-8 Secretion by HRF in BEAS-2B Cells

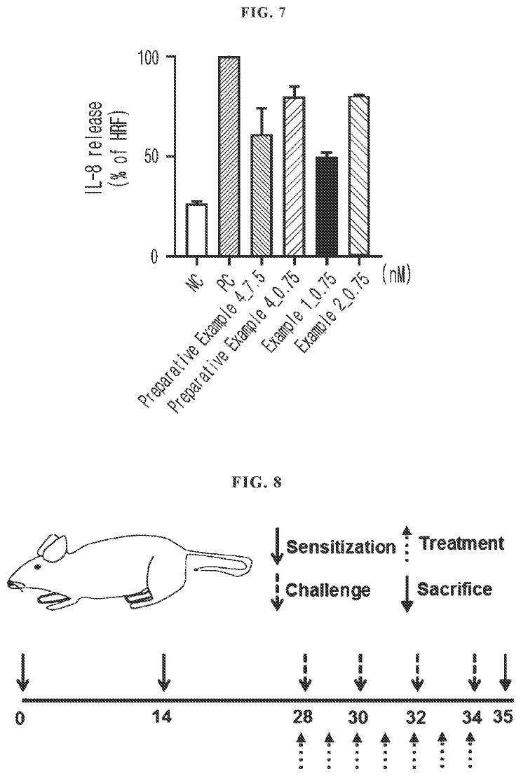

The PEG (5000)-dTBP2 prepared in Preparative Example 4, Example 1, and Example 2 was diluted with 1% penicillin streptomycin/DMEM for each concentration (0, 0.75 nM, 7.5 nM), incubated with 75 nM recombinant HRF at room temperature for 15 minutes, and then treated to BEAS-2B cells grown to about 70% on a 48 well plate. The cells were cultured in a 37° C., 5% CO₂ incubator for 24 hours, and the supernatant was obtained. Then, the released IL-8 was quantified by enzyme immunosorbent detection (Biolegend). The IL-8 enzyme immunosorbent detection was performed according to the protocol of Biolegend Human IL-8 ELISA kit with pre coated plates (431508). 300 μl of wash buffer was added to each well of the plate pre-coated with the IL-8 antibody, and the plate was washed a total of 4 times. Then, 50 μl of the supernatant was added to each well of the plate and left at room temperature for 2 hours. After 2 hours, the supernatant in the plate was removed, and 300 μl of wash buffer was added to each well of the plate. The plate was washed a total of 4 times, and then 100 μl of a detection antibody solution was added to each well of the plate and left at room temperature for 1 hour. Thereafter, 300 μl of wash buffer was added to each well of the plate, and the plate was washed a total of 4 times. Then, 100 μl of Avidin-HRP A solution was added to each well of the plate and left at room temperature for 30 minutes. Finally, 300 μl of wash buffer was added to each well of the plate, and after washing the plate 5 times, 100 μl of substrate solution F was added to each well of the plate and left at room temperature in a dark room. 100 μl of a stop solution was additionally added to each well of the plate, and absorbance was measured at 450 nm and 570 nm. The wash buffer, detection antibody solution, avidin HRP A solution, substrate solution F, and stop solution were all used as reagents included in a kit (431508, Biolegend). The results are shown in FIG. 7.

As a result, as shown in FIG. 7, it was confirmed that the dTBP2 of Preparative Example 1, Example 1 and Example 2 were all effective in the inhibition of IL-8 secretion by HRF in BEAS-2B cells. In particular, it was found that the inhibitory effect of the N-terminal 5K PEG-conjugated dTBP2 according to Example 1 on the IL-8 secretion by HRF was the best.

### <Experimental Example 4> Confirmation of Inhibitory Effect of dTBP2 and N-Terminal 10K PEG-Conjugated dTBP2 on Eosinophil Increase in Bronchoalveolar Lavage Fluid

4-1. Preparation of Ovalbumin-Induced Bronchial Asthma and Rhinitis Mouse Model and Administration of dTBP2 and N-Terminal 10K PEG-Conjugated dTBP2

A bronchial asthma and rhinitis animal model was prepared by the following method. An ovalbumin solution was prepared by dissolving ovalbumin in an aluminum hydroxide solution (1 mg/ml) 28 and 14 days before the start of inducing bronchial asthma and rhinitis, and 200 μl of the solution was intraperitoneally injected into each mouse for sensitization. After 14 days of final sensitization, 20 μl of saline/PBS (normal control; NC) or 20 μl of 10 mg/mL ovalbumin solution (positive control; PC) was instilled into the nasal cavity of each lightly anesthetized mouse 4 times every other day for 8 days to induce bronchial asthma and rhinitis. 200 μl of the dTBP2 according to Preparative Example 4 or the N-terminal 10K PEG-conjugated dTBP2 according to Example 3 (1 mg/kg) was injected intraperitoneally to each mouse while inducing bronchial asthma and rhinitis under the same conditions as the PC group. Dexamethasone (D2915, Sigma Aldrich) was dissolved in sterile tertiary distilled water, and it was administered to each mouse at the concentration of 1 mg/kg the same number of times as the dTBP2 according to Preparative Example 4 and the N-terminal 10K PEG-conjugated dTBP2 according to Example 3 (FIG. 8).

TABLE 1

| | | Sensitization | Instillation | Treatment |
|---|---|---|---|---|
| | Group | (IP) | (IN) | (IP) |
| 1 | NC | PBS | PBS | PBS, IP |
| 2 | PC | OVA/alum | OVA | PBS, IP |
| 3 | dexamethasone | OVA/alum | OVA | 1 mg/kg, IP |
| 4 | Preparative | OVA/alum | OVA | 1 mg/kg, IP |
| | Example 4 | | | |
| 5 | Example 3 | OVA/alum | OVA | 1 mg/kg, IP |

4-2. Confirmation of Inhibitory Effect of dTBP2 and N-Terminal 10K PEG-Conjugated dTBP2 on Eosinophil Increase in Bronchoalveolar Lavage Fluid

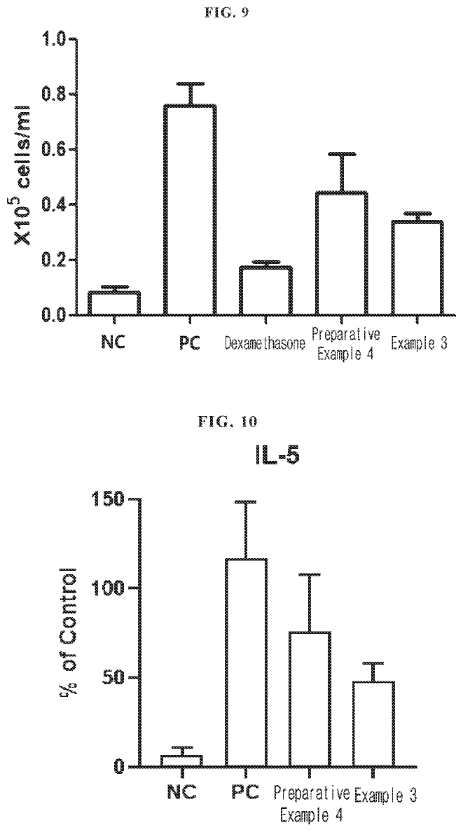

In order to confirm the inhibitory effect of the dTBP2 according to Preparative Example 4 and the N-terminal 10K PEG-conjugated dTBP2 according to Example 3 on eosinophil increase in the bronchial asthma and rhinitis model, the dTBP2 of Preparative Example 4 and Example 3 was administered to the mouse model of Example 4-1 through intraperitoneal injection a total of 8 times daily for 8 days inducing bronchial asthma and rhinitis. After anesthetizing the mouse by injecting a mixture of zoletil (250 mg/kg) and rompun (50 mg/kg) intraperitoneally, a 20-gauge catheter was inserted into the trachea by puncturing the trachea with a knife, and 0.6 mL of PBS at a time was injected-collected three times, and the collection rate was adjusted to 80%. The collected bronchoalveolar lavage fluid was centrifuged at 4° C., 3000 rpm for 10 minutes, and the cell precipitate was resuspended in 0.1 mL of PBS. 20 μL of the suspension was analyzed for the composition of blood cells using a fully automatic hematology analyzer (HEMAVET 950 FS, Drew Scientific, Inc.). The results are shown in FIG. 9.

As a result, as shown in FIG. 9, it was confirmed that eosinophils, which are inflammatory cells, were decreased in both groups treated with the dTBP2 according to Preparative Example 4 and the N-terminal 10K PEG-conjugated dTBP2 according to Example 3 when compared with the positive control group (PC). Particularly, it was confirmed that the dTBP2 according to Preparative Example 4 reduced the number of eosinophils increased to about 0.75×10⁵ cells/mL by the ovalbumin solution by about 40%. It was also confirmed that the N-terminal 10K PEG-conjugated dTBP2 according to Example 3 reduced the increased eosinophils by about 55% with a superior effect. From the above results, it was confirmed that the eosinophil reduction effect could be further improved by pegylation of dTBP2 using the aldehyde-PEG (10000).

### <Experimental Example 5> Confirmation of Inhibitory Effect of dTBP2 and N-Terminal 10K PEG-Conjugated dTBP2 on IL-5 Secretion in Bronchoalveolar Lavage Fluid

In order to confirm the inhibitory effect of the dTBP2 according to Preparative Example 4 and the N-terminal 10K PEG-conjugated dTBP2 according to Example 3 on IL-5 secretion in the bronchial asthma and rhinitis model, the dTBP2 of Preparative Example 4 and Example 3 was administered to the mouse model of Example 4-1 through intraperitoneal injection a total of 8 times daily for 8 days inducing bronchial asthma and rhinitis. After the end of the experiment, the amount of IL-5 in the supernatant obtained by centrifuging bronchoalveolar lavage fluid was measured and quantified by enzyme immunosorbent detection using mouse IL-5 ELISA kit (Biolegend, USA). The results are shown in FIG. 10.

As a result, as shown in FIG. 10, it was confirmed that the dTBP2 according to Preparative Example 4 and the N-terminal 10K PEG-conjugated dTBP2 according to Example 3 effectively inhibited IL-5 secretion in bronchoalveolar lavage fluid. Specifically, the dTBP2 according to Preparative Example 4 reduced IL-5 secretion by about 25%, and the N-terminal 10K PEG-conjugated dTBP2 according to Example 3 reduced IL-5 secretion by about 50%.

Next, in order to confirm whether there is an effect of inhibiting IL-5 secretion even when administered in a small number of times, the dTBP2 of Preparative Example 4 and Example 3 was administered to the mouse model of Example 4-1 through intraperitoneal injection once in 4 days for a total of 2 times for 8 days inducing bronchial asthma and rhinitis. The amount of IL-5 was measured and quantified by the enzyme immunosorbent detection method in the same manner. The results are shown in FIG. 11.

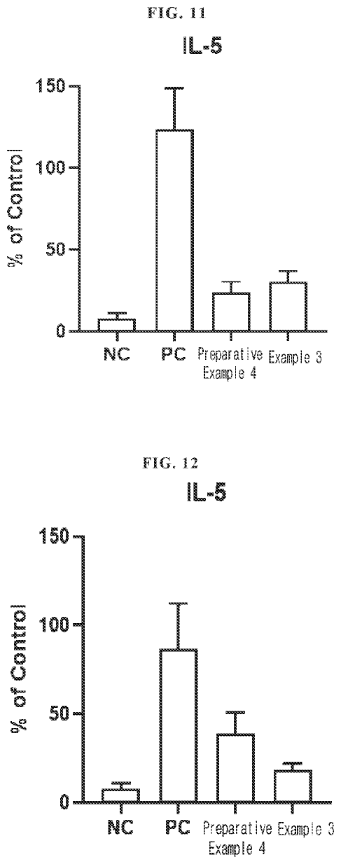

In addition, the dTBP2 of Preparative Example 4 and Example 3 was administered to the mouse model of Example 4-1 through intraperitoneal injection once on the 1st day of 8 days inducing bronchial asthma and rhinitis. The amount of IL-5 was measured and quantified by the enzyme immunosorbent detection method in the same manner. The results are shown in FIG. 12.

As a result, as shown in FIGS. 11 and 12, it was confirmed that the N-terminal 10K PEG-conjugated dTBP2 according to Example 3 was effective in inhibiting IL-5 secretion even when administered only once within the induction period of bronchial asthma and rhinitis.

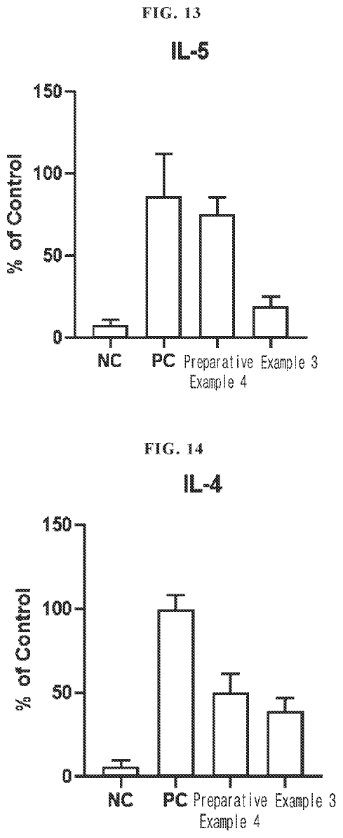

Next, in order to confirm whether there is an inhibitory effect on IL-5 secretion even when administered after inducing bronchial asthma and rhinitis, the dTBP2 of Preparative Example 4 and Example 3 was administered to the mouse model of Example 4-1 through intraperitoneal injection once on the 5th day of 8 days inducing bronchial asthma and rhinitis. The amount of IL-5 was measured and quantified by the enzyme immunosorbent detection method in the same manner. The results are shown in FIG. 13.

As a result, as shown in FIG. 13, it was confirmed that the N-terminal 10K PEG-conjugated dTBP2 according to Example 3 effectively inhibited IL-5 secretion in bronchoalveolar lavage fluid. The dTBP2 according to Preparative Example 4 showed a weak IL-5 inhibitory effect, whereas the N-terminal 10K PEG-conjugated dTBP2 according to Example 3 had an IL-5 secretion inhibitory effect even after only one administration after inducing bronchial asthma and rhinitis. From the above results, it was confirmed that the IL-5 secretion inhibitory effect could be further improved by pegylation of dTBP2 using the aldehyde-PEG (10000).

### <Experimental Example 6> Confirmation of Inhibitory Effect of dTBP2 and N-Terminal 10K PEG-Conjugated dTBP2 on IL-4 Secretion in Bronchoalveolar Lavage Fluid

In order to confirm the inhibitory effect of the dTBP2 according to Preparative Example 4 and the N-terminal 10K PEG-conjugated dTBP2 according to Example 3 on IL-4 secretion in the bronchial asthma and rhinitis model, the dTBP2 of Preparative Example 4 and Example 3 was administered to the mouse model of Example 4-1 through intraperitoneal injection a total of 8 times daily for 8 days inducing bronchial asthma and rhinitis. After the end of the experiment, the amount of IL-4 in the supernatant obtained by centrifuging bronchoalveolar lavage fluid was measured and quantified by enzyme immunosorbent detection using mouse IL-4 ELISA kit (Biolegend, USA). The results are shown in FIG. 14.

As a result, as shown in FIG. 14, it was confirmed that the dTBP2 according to Preparative Example 4 and the N-terminal 10K PEG-conjugated dTBP2 according to Example 3 effectively inhibited IL-4 secretion in bronchoalveolar lavage fluid. Specifically, the dTBP2 according to Preparative Example 4 reduced IL-4 secretion by about 50%, and the N-terminal 10K PEG-conjugated dTBP2 according to Example 3 reduced IL-4 secretion by about 60%.

Next, in order to confirm whether there is an effect of inhibiting IL-4 secretion even when administered in a small number of times, the dTBP2 of Preparative Example 4 and Example 3 was administered to the mouse model of Example 4-1 through intraperitoneal injection once in 4 days for a total of 2 times for 8 days inducing bronchial asthma and rhinitis. The amount of IL-4 was measured and quantified by the enzyme immunosorbent detection method in the same manner. The results are shown in FIG. 15.

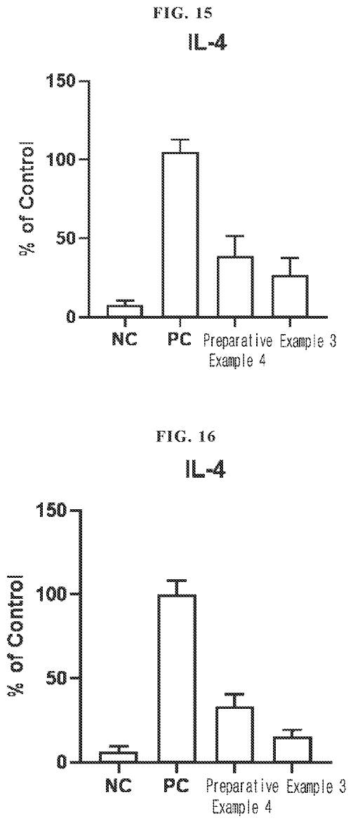

In addition, the dTBP2 of Preparative Example 4 and Example 3 was administered to the mouse model of Example 4-1 through intraperitoneal injection once on the 1st day of 8 days inducing bronchial asthma and rhinitis. The amount of IL-4 was measured and quantified by the enzyme immunosorbent detection method in the same manner. The results are shown in FIG. 16.

As a result, as shown in FIGS. 15 and 16, it was confirmed that the N-terminal 10K PEG-conjugated dTBP2 according to Example 3 was effective in inhibiting IL-4 secretion even when administered only once within the induction period of bronchial asthma and rhinitis.

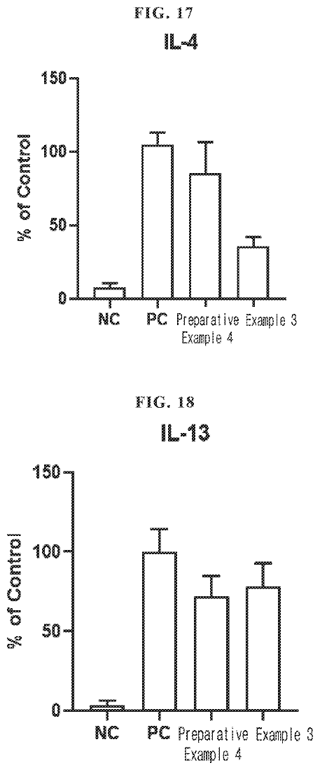

Next, in order to confirm whether there is an inhibitory effect on IL-4 secretion even when administered after inducing bronchial asthma and rhinitis, the dTBP2 of Preparative Example 4 and Example 3 was administered to the mouse model of Example 4-1 through intraperitoneal injection once on the 5th day of 8 days inducing bronchial asthma and rhinitis. The amount of IL-4 was measured and quantified by the enzyme immunosorbent detection method in the same manner. The results are shown in FIG. 17.

As a result, as shown in FIG. 17, it was confirmed that the N-terminal 10K PEG-conjugated dTBP2 according to Example 3 effectively inhibited IL-4 secretion in bronchoalveolar lavage fluid. The dTBP2 according to Preparative Example 4 showed a weak IL-4 inhibitory effect, whereas the N-terminal 10K PEG-conjugated dTBP2 according to Example 3 had an IL-4 secretion inhibitory effect even after only one administration after inducing bronchial asthma and rhinitis. From the above results, it was confirmed that the IL-4 secretion inhibitory effect could be further improved by pegylation of dTBP2 using the aldehyde-PEG (10000).

### <Experimental Example 7> Confirmation of Inhibitory Effect of dTBP2 and N-Terminal 10K PEG-Conjugated dTBP2 on IL-13 Secretion in Bronchoalveolar Lavage Fluid

In order to confirm the inhibitory effect of the dTBP2 according to Preparative Example 4 and the N-terminal 10K PEG-conjugated dTBP2 according to Example 3 on IL-13 secretion in the bronchial asthma and rhinitis model, the dTBP2 of Preparative Example 4 and Example 3 was administered to the mouse model of Example 4-1 through intraperitoneal injection a total of 8 times daily for 8 days inducing bronchial asthma and rhinitis. After the end of the experiment, the amount of IL-13 in the supernatant obtained by centrifuging bronchoalveolar lavage fluid was measured and quantified by enzyme immunosorbent detection using mouse IL-13 ELISA kit (R&D system, USA). The results are shown in FIG. 18.

As a result, as shown in FIG. 18, it was confirmed that the dTBP2 according to Preparative Example 4 and the N-terminal 10K PEG-conjugated dTBP2 according to Example 3 effectively inhibited IL-13 secretion in bronchoalveolar lavage fluid. Specifically, the dTBP2 according to Preparative Example 4 reduced IL-13 secretion by about 30%, and the N-terminal 10K PEG-conjugated dTBP2 according to Example 3 reduced IL-13 secretion by about 20%.

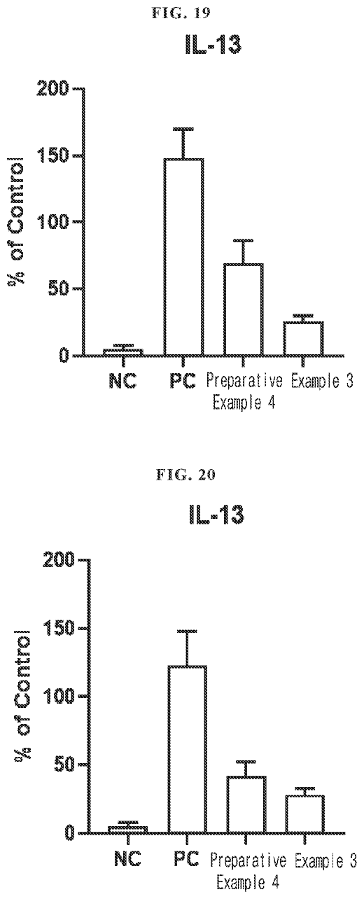

Next, in order to confirm whether there is an effect of inhibiting IL-13 secretion even when administered in a small number of times, the dTBP2 of Preparative Example 4 and Example 3 was administered to the mouse model of Example 4-1 through intraperitoneal injection once in 4 days for a total of 2 times for 8 days inducing bronchial asthma and rhinitis. The amount of IL-13 was measured and quantified by the enzyme immunosorbent detection method in the same manner. The results are shown in FIG. 19.

In addition, the dTBP2 of Preparative Example 4 and Example 3 was administered to the mouse model of Example 4-1 through intraperitoneal injection once on the 1st day of 8 days inducing bronchial asthma and rhinitis. The amount of IL-13 was measured and quantified by the enzyme immunosorbent detection method in the same manner. The results are shown in FIG. 20.

As a result, as shown in FIGS. 19 and 20, it was confirmed that the N-terminal 10K PEG-conjugated dTBP2 according to Example 3 was effective in inhibiting IL-13 secretion even when administered only once within the induction period of bronchial asthma and rhinitis.

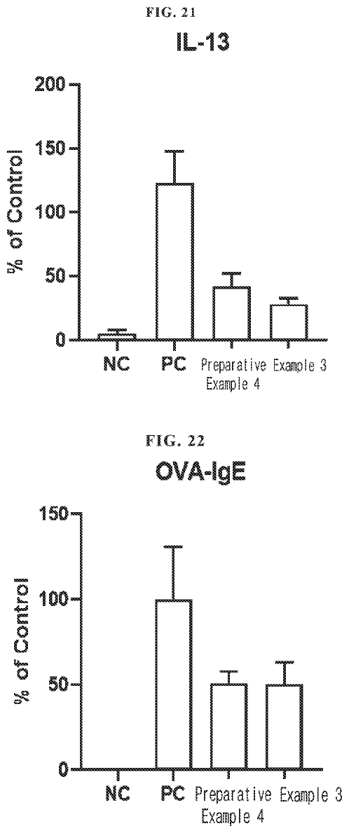

Next, in order to confirm whether there is an inhibitory effect on IL-13 secretion even when administered after inducing bronchial asthma and rhinitis, the dTBP2 of Preparative Example 4 and Example 3 was administered to the mouse model of Example 4-1 through intraperitoneal injection once on the 5th day of 8 days inducing bronchial asthma and rhinitis. The amount of IL-13 was measured and quantified by the enzyme immunosorbent detection method in the same manner. The results are shown in FIG. 21.

As a result, as shown in FIG. 21, it was confirmed that the N-terminal 10K PEG-conjugated dTBP2 according to Example 3 had an IL-13 secretion inhibitory effect even after only one administration after inducing bronchial asthma and rhinitis. From the above results, it was confirmed that the IL-13 secretion inhibitory effect could be further improved by pegylation of dTBP2 using the aldehyde-PEG (10000).

### <Experimental Example 8> Confirmation of Inhibitory Effect of dTBP2 and N-Terminal 10K PEG-Conjugated dTBP2 on Ovalbumin-Specific IgE Secretion in Mouse Plasma

Ovalbumin is an allergy-causing substance mainly contained in egg whites. In order to confirm the inhibitory effect of the dTBP2 according to Preparative Example 4 and the N-terminal 10K PEG-conjugated dTBP2 according to Example 3 on ovalbumin-specific IgE secretion in plasma in the bronchial asthma and rhinitis model, the dTBP2 of Preparative Example 4 and Example 3 was administered to the mouse model of Example 4-1 through intraperitoneal injection a total of 8 times daily for 8 days inducing bronchial asthma and rhinitis. After completion of the experiment, the mouse was anesthetized by injecting a mixture of zoletil (250 mg/kg) and rompun (50 mg/kg) intraperitoneally, and the chest was opened. 0.8 mL blood was collected by puncture of the heart with a 26 gauge 1 mL syringe coated with heparin. The collected blood was centrifuged at 4° C., 3000 rpm for 10 minutes to obtain plasma components. The amount of ovalbumin-specific IgE in the blood serum obtained through the above process was measured and quantified by enzyme immunosorbent detection method using mouse OVA specific IgE kit (Biolegend, USA). The results are shown in FIG. 22.

As a result, as shown in FIG. 22, it was confirmed that the dTBP2 according to Preparative Example 4 and the N-terminal 10K PEG-conjugated dTBP2 according to Example 3 effectively inhibited ovalbumin-specific IgE secretion in plasma. Specifically, the dTBP2 according to Preparative Example 4 reduced ovalbumin-specific IgE secretion by about 50%, and the N-terminal 10K PEG-conjugated dTBP2 according to Example 3 reduced ovalbumin-specific IgE secretion by about 50%.

From the above results, it was confirmed that the dTBP2 and N-terminal 10K PEG-conjugated dTBP2 were very effective in inhibiting ovalbumin-specific IgE secretion in plasma.

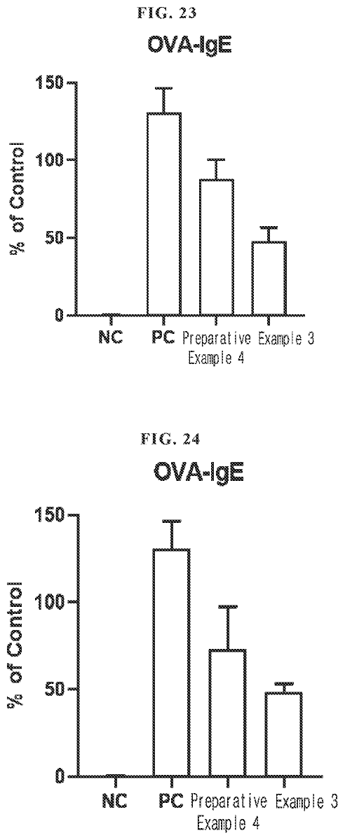

Next, in order to confirm whether there is an effect of inhibiting ovalbumin-specific IgE secretion in plasma even when administered in a small number of times, the dTBP2 of Preparative Example 4 and Example 3 was administered to the mouse model of Example 4-1 through intraperitoneal injection once in 4 days for a total of 2 times for 8 days inducing bronchial asthma and rhinitis. The amount of ovalbumin-specific IgE secretion was measured and quantified by the enzyme immunosorbent detection method in the same manner. The results are shown in FIG. 23.

In addition, the dTBP2 of Preparative Example 4 and Example 3 was administered to the mouse model of Example 4-1 through intraperitoneal injection once on the 1st day of 8 days inducing bronchial asthma and rhinitis. The amount of ovalbumin-specific IgE secretion was measured and quantified by the enzyme immunosorbent detection method in the same manner. The results are shown in FIG. 24.

As a result, as shown in FIGS. 23 and 24, it was confirmed that the N-terminal 10K PEG-conjugated dTBP2 according to Example 3 was effective in inhibiting ovalbumin-specific IgE secretion even when administered only once within the induction period of bronchial asthma and rhinitis. It was also confirmed that the N-terminal 10K PEG-conjugated dTBP2 of Example 3 had better inhibitory ability than the dTBP2 of Preparative Example 4.

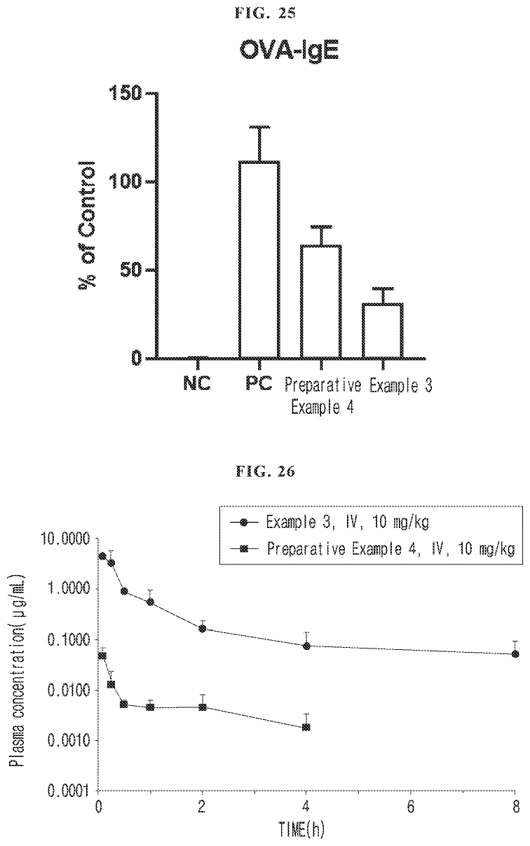

Next, in order to confirm whether there is an inhibitory effect on ovalbumin-specific IgE secretion in plasma even when administered after inducing bronchial asthma and rhinitis, the dTBP2 of Preparative Example 4 and Example 3 was administered to the mouse model of Example 4-1 through intraperitoneal injection once on the 5th day of 8 days inducing bronchial asthma and rhinitis. The amount of ovalbumin-specific IgE secretion was measured and quantified by the enzyme immunosorbent detection method in the same manner. The results are shown in FIG. 25.

As a result, as shown in FIG. 25, it was confirmed that the N-terminal 10K PEG-conjugated dTBP2 according to Example 3 had an ovalbumin-specific IgE secretion inhibitory effect even after only one administration after inducing bronchial asthma and rhinitis. From the above results, it was confirmed that the ovalbumin-specific IgE secretion inhibitory effect could be further improved by pegylation of dTBP2 using the aldehyde-PEG (10000).

### <Experimental Example 9> Pharmacokinetic Analysis of dTBP2 and N-Terminal 10K PEG-Conjugated dTBP2

The dTBP2 according to Preparative Example 4 and the N-terminal 10K PEG-conjugated dTBP2 according to Example 3 were administered to 7-8 weeks old ICR mice that had undergone the acclimatization period at the concentration of 10 mg/kg IV, respectively, and then blood was collected according to a predetermined time. The dTBP2 of Preparative Examples 4 and Example 3 was added to a solution prepared in DMSO:PEG400:DW=5:55:40, and this was administered at the concentration of 5 mL/kg. Blood samples were taken at 0.083, 0.25, 0.5, 1, 2, 4, and 8 hours post injection. The blood was centrifuged at 4° C., 3000 rpm for 10 minutes, to which cold acetonitrile containing internal standard was added in an amount 4 times the plasma component, and then deproteinization was carried out. After centrifugation again at 4° C., 13000 rpm for 10 minutes, the supernatant was collected and analyzed by Agilent 6460 LC-MS/MS. Pharmacokinetic analysis was performed with Phoenix WinNonlin (Pharsight ver 6.4, USA) using a non-compartmental analysis model. The results are shown in FIG. 26, and the pharmacokinetic analysis results were analyzed and shown in Table 2 below.

TABLE 2

| | Preparative Example 4, | Example 3, |
|---|---|---|
| Parameter | IV, 10 mg/kg | IV, 10 mg/kg |
| Tₘₐₓ (h) | NA | NA |
| Cₘₐₓ (μg/mL) | NA | NA |
| T_{1/2} (h) | 1.91 | 3.18 |
| AUCₗₐₛₜ (μg · h/mL) | 0.02 | 2.83 |
| AUC_{∞} (μg · h/mL) | 0.03 | 3.15 |
| CL (L/h/kg) | 292.34 | 3.29 |
| Vₛₛ (L/kg) | 782.86 | 7.37 |
| Fₜ (%) | NA | NA |

NA: not applicable
Tₘₐₓ: time to Cₘₐₓ
Cₘₐₓ: maximum plasma concentration
T_{1/2}: half life
AUCₗₐₛₜ: area of concentration-time curve from beginning to the maximum time at which the concentration can be measured
AUC_{∞}: area of concentration-time curve from beginning to infinity
CL: clearance from plasma
Vₛₛ: steady-state volume of distribution
Fₜ: bioavailability (AUC_{P.O.}/AUC_{I.V.}) × 100

As a result, as shown in FIG. 26, it was confirmed that the stability of the N-terminal 10K PEG-conjugated dTBP2 according to Example 3 in plasma was improved by the dTBP2 according to Preparative Example 4. In addition, as shown in Table 1, it was confirmed that the half-life of the PEGylated dTBP2 of Example 3 was increased by about 1.6 times compared to the dTBP2 of Preparative Example 4. In addition, as shown in FIG. 18, it was confirmed that the initial plasma concentration of the N-terminal 10K PEG-conjugated dTBP2 according to Example 3 was about 1000 times higher than that of the dTBP2 according to Preparative Example 4. From the above results, it was found that the stability was increased by about 1000 times by pegylation of the peptide of Preparative Example 4.

### <Experimental Example 10> Confirmation of Metabolic Stability of 10K PEG-Conjugated dTBP2

In order to evaluate the first-step metabolic stability of the N-terminal 10K PEG-conjugated dTBP2 according to Example 3 in the liver, the following evaluation was performed using mouse and human liver microsomes. After incubating the mouse and human liver microsomes diluted with 0.5 M potassium phosphate buffer (pH 7.4) at 37° C. for 5 minutes, NADPH for activating the metabolic enzyme system was reacted with the peptide of Example 3 at 37° C. for 30 minutes. Then, the amount of the peptide of Example 3 remaining was analyzed. Upon completion of the reaction, cold acetonitrile containing internal standard was added, and then deproteinization was carried out. The reactant was centrifuged at 4° C., 4000 rpm for 15 minutes, and the supernatant was analyzed by LC-MS/MS. The system was verified with buspirone, a reference material. The results are shown in Table 3.

TABLE 3

| Compound | Mouse (%) | Human (%) |
|---|---|---|
| Example 3 | 75.26 | 75.77 |
| Buspirone | 0.10 | 6.15 |

In the evaluation of the experiment, the microsome stability evaluation criteria according to the % value, which is the result of the reaction experiment for 30 minutes, are as follows (R. SCOTT OBACH, Prediction of human clearance of twenty-nine drugs form hepatic microsomal intrinsic clearance data: An examination of in vitro half-life approach and nonspecific binding to microsomes, 1999, 27, (11): 1350-59).

>90%: very stable compound with a half-life longer than 3 hours

70˜90%: stable compound with a half-life of 1 to 3 hours

50˜70%: relatively stable compound with a half-life of 30 to 60 minutes

30˜50%: relatively unstable compound with a half-life of 15 to 30 minutes

<30%: unstable compound with a half-life of less than 15 minutes

Referring to the above evaluation criteria, the N-terminal 10K PEG-conjugated dTBP2 according to Example 3 of the present invention was confirmed to be a stable compound with a half-life of about 1 to 3 hours since the remaining drug was more than 75% after 30 minutes in both mice and humans. Therefore, since the peptide can exert a drug effect with high stability in vivo, the number of administration can be reduced, so that it can be effectively used as a drug.

As mentioned above, the present invention has been described in detail through the preferred preparative examples, examples and experimental examples, but the scope of the present invention is not limited to the specific examples, and should be interpreted by the appended claims. In addition, those of ordinary skill in the art should understand that many modifications and variations are possible without departing from the scope of the present invention.

1.-23. (canceled)

24. A PEGylated HRF-binding peptide in which polyethylene glycol is bound to the HRF peptide comprising a sequence of amino acids wherein the first amino acid is selected from the group consisting of A, L and W; the second amino acid is selected from the group consisting of V, Y, E and A; the third amino acid is selected from the group consisting of T, V, F and A; the fourth amino acid is selected from the group consisting of Y, P and A; the fifth amino acid is selected from the group consisting of P, G and K; the sixth amino acid is selected from the group consisting of A, L, S and W; and the seventh amino acid consists of a sequence of amino acids selected from the group consisting of A, P and M.

25. The PEGylated HRF-binding peptide according to claim 24, wherein the polyethylene glycol or the derivative thereof has a molecular weight of 1 kDa to 50 kDa.

26. The PEGylated HRF-binding peptide according to claim 24, wherein the polyethylene glycol is covalently bound to a carboxyl group or an amino group of the HRF-binding peptide.

27. The PEGylated HRF-binding peptide according to claim 24, wherein the polyethylene glycol has a functional group including an aldehyde group, a carboxyl group, an amino group or a hydrazide group bound to a terminal thereof.

28. The PEGylated HRF-binding peptide according to claim 24, wherein the polyethylene glycol or the derivative thereof is bound to the N-terminal or C-terminal of the peptide.

29. The PEGylated HRF-binding peptide according to claim 24, wherein the HRF-binding peptide consists of the amino acid sequence W-Y-V-Y-P-S-M.

30. The PEGylated HRF-binding peptide according to claim 24, wherein the PEGylated HRF-binding peptide has an inhibitory effect on the secretion of IL-8.

31. The PEGylated HRF-binding peptide according to claim 24, wherein the PEGylated HRF-binding peptide has an inhibitory effect on the increase of eosinophils.

32. The PEGylated HRF-binding peptide according to claim 24, wherein the PEGylated HRF-binding peptide has an inhibitory effect on the secretion of IL-5.

33. The PEGylated HRF-binding peptide according to claim 24, wherein the PEGylated HRF-binding peptide has an inhibitory effect on the secretion of IL-4.

34. The PEGylated HRF-binding peptide according to claim 24, wherein the PEGylated HRF-binding peptide has an inhibitory effect on the secretion of IL-13.

35. The PEGylated HRF-binding peptide according to claim 24, wherein the PEGylated HRF-binding peptide has an inhibitory effect on the ovalbumin-specific secretion of IgE.

36. A method for preventing or treating allergy comprising a step of administering the PEGylated HRF-binding peptide of claim 24 to an individual or subject in need thereof.

37. The method according to claim 36, wherein the allergy is asthma, rhinitis, atopy, urticaria, anaphylaxis, allergic bronchiectasis, allergy caused by food, drug, pollen, mold or insects, allergic conjunctivitis, hay fever, cold urticaria, or atopic dermatitis.

38. A method for preventing or treating malaria comprising a step of administering the PEGylated HRF-binding peptide of claim 24 to an individual or subject in need thereof.

39. The method according to claim 38, wherein the malaria is Plasmodium falciparum, Plasmodium vivax, Plasmodium ovale, or Plasmodium malariae.

40. A method for preventing or treating autoimmune disease comprising a step of administering the PEGylated HRF-binding peptide of claim 24 to an individual or subject in need thereof.

41. The method according to claim 40, wherein the autoimmune disease is rheumatoid arthritis, Sjogrean's disease, systemic sclerosis, polymyositis, systemic angitis, mixed connective tissue disease, Crohn's disease, Hashimoto's disease, Grave's disease, Goodpasture's syndrome, Guillain-Barre syndrome, idiopathic thrombocytopenic purpura, irritable bowel syndrome, myasthenia gravis, narcolepsy, pemphigus vulgaris, pernicious anemia, primary biliary cirrhosis, ulcerative colitis, vasculitis, Wegener's granulomatosis, psoriasis, alopecia areata, rheumatic fever, systemic lupus erythematosus, or multiple scleorosis.

42. A method for preventing or treating acute or chronic inflammatory disease comprising a step of administering the PEGylated HRF-binding peptide of claim 24 to an individual or subject in need thereof.

43. The method according to claim 42, wherein the acute or chronic inflammatory disease is conjunctivitis, periodontitis, rhinitis, otitis media, pharyngitis, tonsillitis, dermatitis, gastritis, colitis, ankylosing spondylitis, psoriatic arthritis, osteoarthritis, rheumatoid arthritis, periarthritis, tendinitis, xerosis, periostitis, myositis, hepatitis, cystitis, nephritis, pneumonia, gastric ulcer, Crohn's disease, Sjogrean's disease, gout, fibromyalgia, lupus, bursitis, or systemic lupus erythematodes.

44. A method for preventing or treating hypertension comprising a step of administering the PEGylated HRF-binding peptide of claim 24 to an individual or subject in need thereof.

45. A method for preventing or treating cancer comprising a step of administering the PEGylated HRF-binding peptide of claim 24 to an individual or subject in need thereof.

46. The method according to claim 45, wherein the cancer is oral cancer, liver cancer, stomach cancer, colon cancer, breast cancer, lung cancer, bone cancer, pancreatic cancer, skin cancer, head and neck cancer, skin cancer, cervical cancer, ovarian cancer, colorectal cancer, small intestine cancer, rectal cancer, fallopian tube carcinoma, perianal cancer, endometrial carcinoma, vaginal carcinoma, vulvar carcinoma, Hodgkin's disease, esophageal cancer, lymph adenocarcinoma, bladder cancer, gallbladder cancer, endocrine adenocarcinoma, thyroid cancer, parathyroid cancer, adrenal cancer, soft tissue sarcoma, urethral cancer, penile cancer, prostate cancer, chronic leukemia, acute leukemia, lymphocytic lymphoma, renal cancer, ureter cancer, renal cell carcinoma, renal pelvic carcinoma, central nervous system tumor, primary central nervous system lymphoma, spinal cord tumor, brainstem glioma or pituitary adenoma.
